(19) United States
(12) Patent Application Publication
(10) Pub. No.: US 2023/0289534 A1
(43) Pub. Date: Sep. 14, 2023

(54) INFORMATION PROCESSING APPARATUS, INFORMATION PROCESSING METHOD, AND INFORMATION PROCESSING PROGRAM

(71) Applicant: FUJIFILM Corporation, Tokyo (JP)

(72) Inventor: Takuya YUZAWA, Tokyo (JP)

(73) Assignee: FUJIFILM Corporation, Tokyo (JP)

(21) Appl. No.: 18/177,727

(22) Filed: Mar. 2, 2023

(30) Foreign Application Priority Data
Mar. 8, 2022 (JP) 2022-035613

(51) Int. Cl. G06F 40/30; G06T 11/20; G16H 30/40

(52) U.S. Cl. CPC G06F 40/30; G06T 11/203; G16H 30/40; G06T 2210/41

(57) ABSTRACT

An information processing apparatus comprising at least one processor, wherein the at least one processor is configured to: acquire a plurality of measurement values measured from the same subject at a plurality of different points in time; acquire a sentence corresponding to the measurement value; and select at least some of the plurality of measurement values based on a phrase related to the measurement value included in the sentence.

### CROSS-REFERENCE TO RELATED APPLICATIONS

This application claims priority from Japanese Application No. 2022-035613, filed on Mar. 8, 2022, the entire disclosure of which is incorporated herein by reference.

### BACKGROUND

### Technical Field

The present disclosure relates to an information processing apparatus, an information processing method, and an information processing program.

### Related Art

In the related art, image diagnosis is performed using medical images obtained by imaging apparatuses such as computed tomography (CT) apparatuses and magnetic resonance imaging (MRI) apparatuses. In addition, medical images are analyzed via computer aided detection/diagnosis (CAD) using a discriminator in which learning is performed by deep learning or the like, and regions of interest including structures, lesions, and the like included in the medical images are detected and/or diagnosed. The medical images and analysis results via CAD are transmitted to a terminal of a healthcare professional such as a radiologist who interprets the medical images. The healthcare professional such as a radiologist interprets the medical image by referring to the medical image and analysis result using his or her own terminal and creates an interpretation report.

In addition, various methods have been proposed to support the creation of interpretation reports in order to reduce the burden of the interpretation work. For example, JP2019-153250A discloses a technique for creating an interpretation report based on a keyword input by a radiologist and an analysis result of a medical image. In the technique described in JP2019-153250A, a sentence to be included in the interpretation report is created by using a recurrent neural network trained to generate a sentence from input characters.

Further, for example, in regular health checkups and post-treatment follow-up observations, the same subject may be examined a plurality of times and data on various measurement values such as a size of a lesion may be accumulated at a plurality of points in time. Various methods have been proposed for making it possible to check changes over time in measurement values by using the plurality of accumulated measurement values. For example, JP2018-181340A discloses presenting medical data in forms such as plots and graphs, and highlighting the medical data in response to user-input features.

Incidentally, in a case where a creator and a viewer of the interpretation report actually check a plurality of measurement values, they may have paid attention to some measurement values instead of checking all the measurement values for the same subject. For example, in a case where a measurement value does not cause any problem for a while from the beginning, but changes in the most recent few times such that it suddenly deteriorates, the measurement values in the most recent few times have sometimes been paid attention to. Therefore, there is a demand for a technique that enables selective checking of some measurement values among a plurality of measurement values.

### SUMMARY

The present disclosure provides an information processing apparatus, an information processing method, and an information processing program capable of supporting creation of medical documents.

According to a first aspect of the present disclosure, there is provided an information processing apparatus comprising at least one processor, in which the processor is configured to: acquire a plurality of measurement values measured from the same subject at a plurality of different points in time; acquire a sentence corresponding to the measurement value; and select at least some of the plurality of measurement values based on a phrase related to the measurement value included in the sentence.

In the first aspect, the processor may be configured to select at least some of the plurality of measurement values based on a phrase that expresses a change over time in the measurement value included in the sentence.

In the first aspect, time information indicating a point in time of measurement may be added to the measurement value, and the processor may be configured to: create a plot diagram including the at least some selected measurement values using the measurement value and the time information as variables; and cause a display to display the plot diagram.

In the first aspect, the processor may be configured to, in a case where an instruction is received: create the plot diagram including all of the plurality of acquired measurement values; and cause the display to display the plot diagram.

In the first aspect, time information indicating a point in time of measurement may be added to the measurement value, and the processor may be configured to select the measurement value to which the time information indicating the point in time of measurement determined based on the phrase related to the measurement value is added.

In the first aspect, the processor may be configured to select at least some of the plurality of measurement values according to the number of measurement values determined based on the phrase related to the measurement value.

In the first aspect, the processor may be configured to select at least some of the plurality of measurement values based on a phrase that expresses a disease name included in the sentence.

In the first aspect, the processor may be configured to select at least some of the plurality of measurement values based on a phrase that expresses a purpose of examination included in the sentence.

In the first aspect, the processor may be configured to determine whether to select the measurement value based on a result of comparison between the measurement value included in the sentence and a predetermined threshold value.

In the first aspect, the processor may be configured to determine whether to select at least two measurement values included in the sentence based on a result of comparison between a difference between the at least two measurement values and a predetermined threshold value.

In the first aspect, the processor may be configured to select the measurement value that satisfies a predetermined condition from among the plurality of measurement values.

In the first aspect, the processor may be configured to, in a case where a difference between at least two measurement values included in the plurality of measurement values satisfies a predetermined condition, select the at least two measurement values.

In the first aspect, time information indicating a point in time of measurement may be added to the measurement value, and the processor may be configured to select at least some of the plurality of measurement values that are continuous in time series order.

In the first aspect, time information indicating a point in time of measurement may be added to the measurement value, and the processor may be configured to select at least some of the plurality of measurement values that are discrete in time series order.

In the first aspect, the measurement value may be at least one of a size of a lesion or a signal value at a part of the lesion in a medical image obtained by imaging the lesion.

According to a second aspect of the present disclosure, there is provided an information processing method comprising: acquiring a plurality of measurement values measured from the same subject at a plurality of different points in time; acquiring a sentence corresponding to the measurement value; and selecting at least some of the plurality of measurement values based on a phrase related to the measurement value included in the sentence.

According to a third aspect of the present disclosure, there is provided an information processing program for causing a computer to execute a process comprising: acquiring a plurality of measurement values measured from the same subject at a plurality of different points in time; acquiring a sentence corresponding to the measurement value; and selecting at least some of the plurality of measurement values based on a phrase related to the measurement value included in the sentence.

The information processing apparatus, the information processing method, and the information processing program according to the aspects of the present disclosure can support the creation of medical documents.

### BRIEF DESCRIPTION OF THE DRAWINGS

FIG. 1 is a diagram showing an example of a schematic configuration of an information processing system.

FIG. 2 is a diagram showing an example of a medical image.

FIG. 3 is a diagram showing an example of a medical image.

FIG. 4 is a block diagram showing an example of a hardware configuration of an information processing apparatus.

FIG. 5 is a block diagram showing an example of a functional configuration of the information processing apparatus.

FIG. 6 is a diagram showing an example of a plurality of measurement values.

FIG. 7 is a diagram showing an example of a plot diagram including the plurality of measurement values.

FIG. 8 is a diagram showing an example of a screen according to a first example.

FIG. 9 is a diagram showing an example of a screen according to a second example.

FIG. 10 is a diagram showing an example of a screen according to a third example.

FIG. 11 is a diagram showing an example of a screen according to a fourth example.

FIG. 12 is a diagram showing an example of a screen according to a fifth example.

FIG. 13 is a flowchart showing an example of information processing.

### DETAILED DESCRIPTION

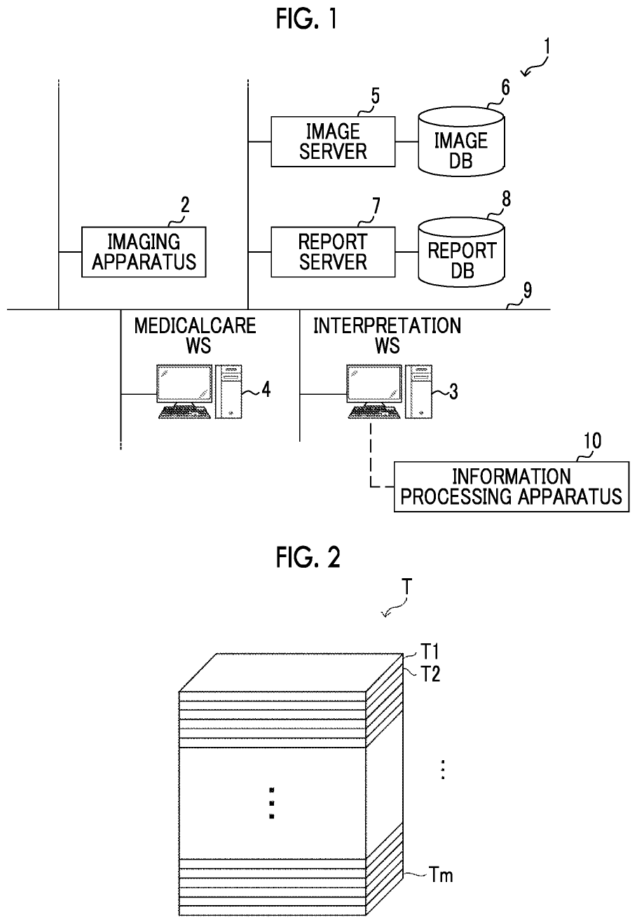

Hereinafter, an embodiment of the present disclosure will be described with reference to the drawings. First, a configuration of an information processing system 1 to which an information processing apparatus of the present disclosure is applied will be described. FIG. 1 is a diagram showing a schematic configuration of the information processing system 1. The information processing system 1 shown in FIG. 1 performs imaging of an examination target part of a subject and storing of a medical image acquired by the imaging based on an examination order from a doctor in a medical department using a known ordering system. In addition, the information processing system 1 performs an interpretation work of a medical image and creation of an interpretation report by a radiologist and viewing of the interpretation report by a doctor of a medical department that is a request source.

As shown in FIG. 1, the information processing system 1 includes an imaging apparatus 2, an interpretation work station (WS) 3 that is an interpretation terminal, a medical care WS 4, an image server 5, an image database (DB) 6, a report server 7, and a report DB 8. The imaging apparatus 2, the interpretation WS 3, the medical care WS 4, the image server 5, the image DB 6, the report server 7, and the report DB 8 are connected to each other via a wired or wireless network 9 in a communicable state.

Each apparatus is a computer on which an application program for causing each apparatus to function as a component of the information processing system 1 is installed. The application program may be recorded on, for example, a recording medium, such as a digital versatile disc read only memory (DVD-ROM) or a compact disc read only memory (CD-ROM), and distributed, and be installed on the computer from the recording medium. In addition, the application program may be stored in, for example, a storage apparatus of a server computer connected to the network 9 or in a network storage in a state in which it can be accessed from the outside, and be downloaded and installed on the computer in response to a request.

The imaging apparatus 2 is an apparatus (modality) that generates a medical image T showing a diagnosis target part of the subject by imaging the diagnosis target part. Examples of the imaging apparatus 2 include a simple X-ray imaging apparatus, a computed tomography (CT) apparatus, a magnetic resonance imaging (MRI) apparatus, a positron emission tomography (PET) apparatus, an ultrasound diagnostic apparatus, an endoscope, a fundus camera, and the like. The medical image generated by the imaging apparatus 2 is transmitted to the image server 5 and is saved in the image DB 6.

The interpretation WS 3 is a computer used by, for example, a healthcare professional such as a radiologist of a radiology department to interpret a medical image and to create an interpretation report, and encompasses an information processing apparatus 10 according to the present embodiment. In the interpretation WS 3, a viewing request for a medical image to the image server 5, various image processing for the medical image received from the image server 5, display of the medical image, and input reception of a sentence regarding the medical image are performed. In the interpretation WS 3, an analysis process for medical images, support for creating an interpretation report based on the analysis result, a registration request and a viewing request for the interpretation report to the report server 7, and display of the interpretation report received from the report server 7 are performed. The above processes are performed by the interpretation WS 3 executing software programs for respective processes.

The medical care WS 4 is a computer used by, for example, a healthcare professional such as a doctor in a medical department to observe a medical image in detail, view an interpretation report, create an electronic medical record, and the like, and is configured to include a processing apparatus, a display apparatus such as a display, and an input apparatus such as a keyboard and a mouse. In the medical care WS 4, a viewing request for the medical image to the image server 5, display of the medical image received from the image server 5, a viewing request for the interpretation report to the report server 7, and display of the interpretation report received from the report server 7 are performed. The above processes are performed by the medical care WS 4 executing software programs for respective processes.

The image server 5 is a general-purpose computer on which a software program that provides a function of a database management system (DBMS) is installed. The image server 5 is connected to the image DB 6. The connection form between the image server 5 and the image DB 6 is not particularly limited, and may be a form connected by a data bus, or a form connected to each other via a network such as a network attached storage (NAS) and a storage area network (SAN).

The image DB 6 is realized by, for example, a storage medium such as a hard disk drive (HDD), a solid state drive (SSD), and a flash memory. In the image DB 6, the medical image acquired by the imaging apparatus 2 and accessory information attached to the medical image are registered in association with each other.

The accessory information may include, for example, identification information such as an image identification (ID) for identifying a medical image, a tomographic ID assigned to each tomographic image included in the medical image, a subject ID for identifying a subject, and an examination ID for identifying an examination. In addition, the accessory information may include, for example, information related to imaging such as an imaging method, an imaging condition, and an imaging date and time related to imaging of a medical image. The “imaging method” and “imaging condition” are, for example, a type of the imaging apparatus 2, an imaging part, an imaging protocol, an imaging sequence, an imaging method, the presence or absence of use of a contrast medium, a slice thickness in tomographic imaging, and the like. In addition, the accessory information may include information related to the subject such as the name, date of birth, age, and gender of the subject. In addition, the accessory information may include information regarding the imaging purpose of the medical image.

In a case where the image server 5 receives a request to register a medical image from the imaging apparatus 2, the image server 5 prepares the medical image in a format for a database and registers the medical image in the image DB 6. In addition, in a case where the viewing request from the interpretation WS 3 and the medical care WS 4 is received, the image server 5 searches for a medical image registered in the image DB 6 and transmits the searched for medical image to the interpretation WS 3 and to the medical care WS 4 that are viewing request sources.

The report server 7 is a general-purpose computer on which a software program that provides a function of a database management system is installed. The report server 7 is connected to the report DB 8. The connection form between the report server 7 and the report DB 8 is not particularly limited, and may be a form connected by a data bus or a form connected via a network such as a NAS and a SAN.

The report DB 8 is realized by, for example, a storage medium such as an HDD, an SSD, and a flash memory. In the report DB 8, an interpretation report created in the interpretation WS 3 is registered.

Further, in a case where the report server 7 receives a request to register the interpretation report from the interpretation WS 3, the report server 7 prepares the interpretation report in a format for a database and registers the interpretation report in the report DB 8. Further, in a case where the report server 7 receives the viewing request for the interpretation report from the interpretation WS 3 and the medical care WS 4, the report server 7 searches for the interpretation report registered in the report DB 8, and transmits the searched for interpretation report to the interpretation WS 3 and to the medical care WS 4 that are viewing request sources.

The network 9 is, for example, a network such as a local area network (LAN) and a wide area network (WAN). The imaging apparatus 2, the interpretation WS 3, the medical care WS 4, the image server 5, the image DB 6, the report server 7, and the report DB 8 included in the information processing system 1 may be disposed in the same medical institution, or may be disposed in different medical institutions or the like. Further, the number of each apparatus of the imaging apparatus 2, the interpretation WS 3, the medical care WS 4, the image server 5, the image DB 6, the report server 7, and the report DB 8 is not limited to the number shown in FIG. 1, and each apparatus may be composed of a plurality of apparatuses having the same functions.

FIG. 2 is a diagram schematically showing an example of a medical image acquired by the imaging apparatus 2. The medical image T shown in FIG. 2 is, for example, a CT image consisting of a plurality of tomographic images T1 to Tm (m is 2 or more) representing tomographic planes from the head to the lumbar region of one subject (human body).

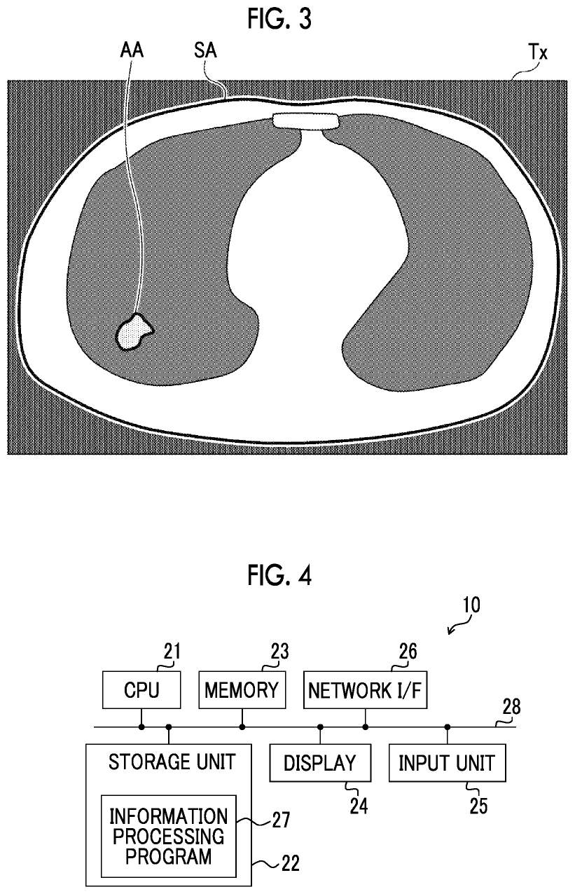

FIG. 3 is a diagram schematically showing an example of one tomographic image Tx out of the plurality of tomographic images T1 to Tm. The tomographic image Tx shown in FIG. 3 represents a tomographic plane including a lung. Each of the tomographic images T1 to Tm may include a region SA of a structure showing various organs and viscera of the human body (for example, lungs, livers, and the like), various tissues constituting various organs and viscera (for example, blood vessels, nerves, muscles, and the like), and the like. In addition, each tomographic image may include a region AA of an abnormal shadow showing lesions such as, for example, nodules, tumors, injuries, defects, and inflammation. In the tomographic image Tx shown in FIG. 3, the lung region is the region SA of the structure, and the nodule region is the region AA of the abnormal shadow. A single tomographic image may include regions SA of a plurality of structures and/or regions AA of a plurality of abnormal shadows.

Incidentally, for example, in regular health checkups and post-treatment follow-up observations, the same subject may be examined a plurality of times and data on various measurement values such as a size of a lesion may be accumulated at a plurality of points in time. The information processing apparatus 10 according to the present embodiment has a function of supporting the creation of medical documents by selectively presenting a measurement value assumed to attract the user's attention among measurement values at a plurality of points in time. The information processing apparatus 10 will be described below. As described above, the information processing apparatus 10 is encompassed in the interpretation WS 3.

First, with reference to FIG. 4, an example of a hardware configuration of the information processing apparatus 10 according to the present embodiment will be described. As shown in FIG. 4, the information processing apparatus 10 includes a central processing unit (CPU) 21, a non-volatile storage unit 22, and a memory 23 as a temporary storage area. Further, the information processing apparatus 10 includes a display 24 such as a liquid crystal display, an input unit 25 such as a keyboard and a mouse, and a network interface (I/F) 26. The network I/F 26 is connected to the network 9 and performs wired or wireless communication. The CPU 21, the storage unit 22, the memory 23, the display 24, the input unit 25, and the network I/F 26 are connected to each other via a bus 28 such as a system bus and a control bus so that various types of information can be exchanged.

The storage unit 22 is realized by, for example, a storage medium such as an HDD, an SSD, and a flash memory. An information processing program 27 in the information processing apparatus 10 is stored in the storage unit 22. The CPU 21 reads out the information processing program 27 from the storage unit 22, loads the read-out program into the memory 23, and executes the loaded information processing program 27. The CPU 21 is an example of a processor of the present disclosure. As the information processing apparatus 10, for example, a personal computer, a server computer, a smartphone, a tablet terminal, a wearable terminal, or the like can be appropriately applied.

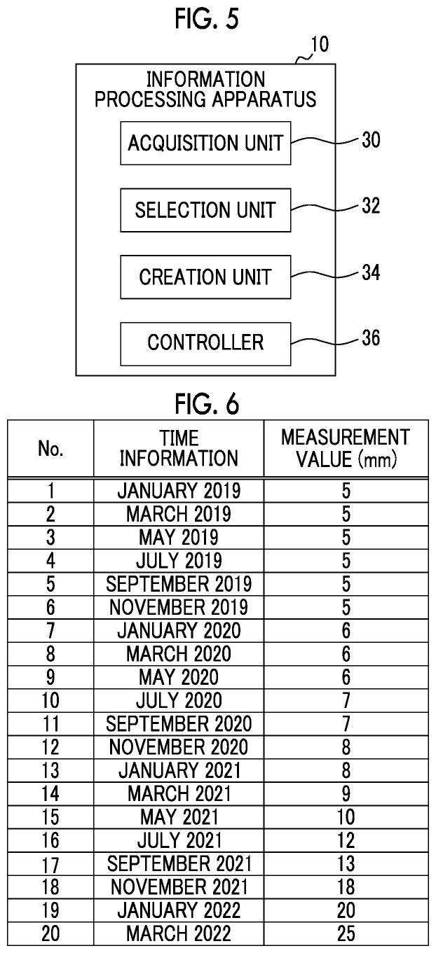

Next, with reference to FIGS. 5 to 12, an example of a functional configuration of the information processing apparatus 10 according to the present embodiment will be described. As shown in FIG. 5, the information processing apparatus 10 includes an acquisition unit 30, a selection unit 32, a creation unit 34, and a controller 36. In a case where the CPU 21 executes the information processing program 27, the CPU 21 functions as the acquisition unit 30, the selection unit 32, the creation unit 34, and the controller 36.

The acquisition unit 30 acquires a plurality of measurement values measured from the same subject at a plurality of different points in time. The measurement value may be, for example, at least one of a size of a lesion or a signal value at the lesion part in a medical image obtained by imaging the lesion. The size of a lesion is represented, for example, by a major axis, a minor axis, an area, a volume, or the like of the region AA of the abnormal shadow included in the medical image Tx. The signal value is represented, for example, by a pixel value of the region AA of the abnormal shadow included in the medical image Tx, a CT value in units of HU, or the like.

Specifically, the acquisition unit 30 may acquire a plurality of medical images captured at a plurality of different points in time from the image server 5, and may acquire measurement values by performing image analysis on the plurality of medical images. For example, the acquisition unit 30 may derive a measurement value based on an image feature amount derived using a learning model such as a convolutional neural network (CNN), which has been trained in advance so that the input is a medical image and the output is an image feature amount of the medical image.

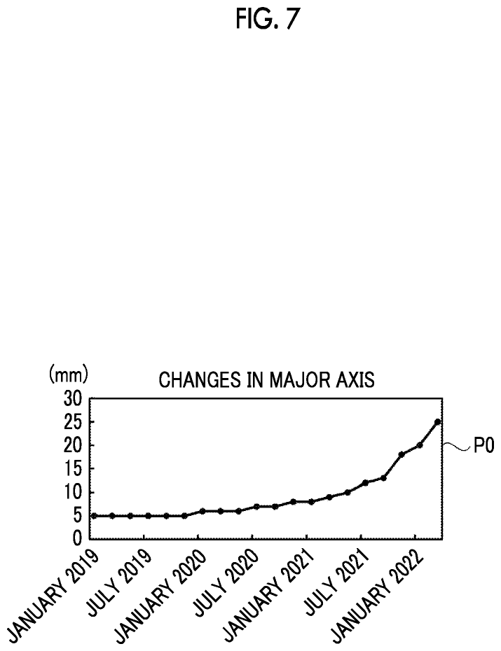

FIG. 6 shows measurement values representing the major axis of the region AA of the abnormal shadow as an example of a plurality of measurement values. As shown in FIG. 6, time information indicating a point in time of measurement is added to the measurement value. The time information may be any information that can arrange a plurality of measurement values in the order in which they were measured (that is, in time series order), and may be, for example, information indicating the date and time as shown in FIG. 6, or information indicating the number of measurements. That is, the measurement values are time-series data. FIG. 7 shows a plot diagram P0 including all of the plurality of measurement values shown in FIG. 6. The plot diagram P0 is a line graph with the vertical axis representing the measurement value and the horizontal axis representing the time information added to the measurement value.

The acquisition unit 30 also acquires sentences corresponding to the plurality of acquired measurement values. Sentences corresponding to measurement values are, specifically, sentences that can include descriptions related to measurement values, such as changes over time in measurement values, results of comparison of measurement values with reference values, names of diseases diagnosed based on measurement values, and purposes of examination. Such sentences may be, for example, comments on findings and other accessory information described in the interpretation report.

Specifically, the acquisition unit 30 may acquire a medical image from the image server 5, generate a comment on findings corresponding to the measurement value from the medical image by machine learning, and acquire the comment on findings as a sentence corresponding to the measurement value. As a method of generating a comment on findings using machine learning, for example, a method using a recurrent neural network described in JP2019-153250A can be appropriately applied. Alternatively, for example, the acquisition unit 30 may generate a comment on findings by a known method of generating a comment on findings using a predetermined template, and acquire the comment on findings as a sentence corresponding to the measurement value.

The selection unit 32 specifies phrases related to the measurement value included in the sentence acquired by the acquisition unit 30. “Phrases related to measurement values” include, for example, phrases that express changes over time in measurement values, phrases that express the names of diseases diagnosed from measurement values, phrases that express the purpose of examination, and phrases that express absolute values of measurement values. As a method for specifying phrases from a sentence, a known named entity extraction method using a natural language processing model such as bidirectional encoder representations from transformers (BERT) can be appropriately applied.

In addition, the selection unit 32 selects at least some of the plurality of measurement values acquired by the acquisition unit 30 based on a specified phrase related to the measurement value. Specifically, the selection unit 32 may select a measurement value to which time information indicating a point in time of measurement determined based on a phrase related to the measurement value is added. Moreover, the selection unit 32 may select at least some of the plurality of measurement values according to the number of measurement values determined based on phrases related to the measurement values. Which part of the plurality of measurement values is to be selected may be determined in advance for each phrase and stored in the storage unit 22, for example.

The creation unit 34 creates a plot diagram including at least some of the measurement values selected by the selection unit 32 using the measurement values and the time information added to the measurement values as variables. The controller 36 controls the display 24 to display the plot diagram created by the creation unit 34. An example of how the selection unit 32 selects some of the plurality of measurement values and how the creation unit 34 creates a plot diagram will be described below in first to tenth examples.

### First Example

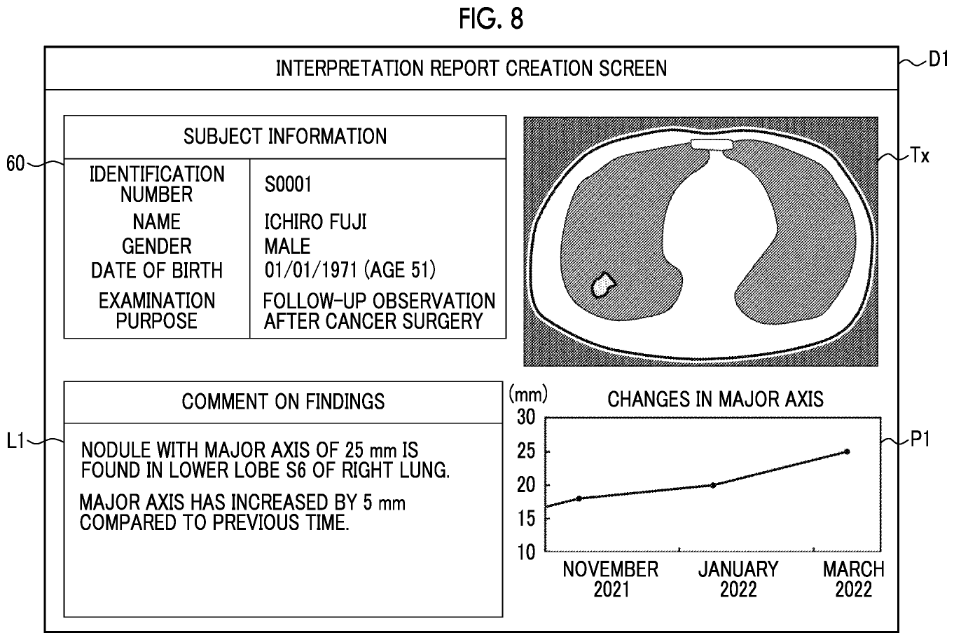

A first example will be described with reference to FIG. 8. FIG. 8 is an example of a screen D1 for creating an interpretation report, which is displayed on the display 24 by the controller 36. The screen D1 includes subject information 60, a comment on findings L1, a medical image Tx, and a plot diagram P1. The comment on findings L1 is an example of a sentence corresponding to a measurement value acquired by the acquisition unit 30. The medical image Tx is acquired from the image server 5 by the acquisition unit 30. The subject information 60 is information indicating the subject ID, the name, date of birth, age, and gender of the subject, and examination purpose, which are included in the accessory information of the medical image Tx acquired by the acquisition unit 30.

The comment on findings L1 includes a phrase that expresses changes over time in measurement values, such as “The major axis has increased by 5 mm compared to the previous time”. In a case where the user checks the measurement value corresponding to this phrase, it is assumed that the user will pay attention to the most recent two to three measurement values among all the plurality of measurement values (see FIG. 7).

Therefore, the selection unit 32 specifies a phrase that expresses changes over time in the measurement values included in the comment on findings L1. In addition, the selection unit 32 selects at least some of the plurality of measurement values acquired by the acquisition unit 30 based on a specified phrase that expresses changes over time in the measurement values. For example, as shown in the plot diagram P1 of FIG. 8, the selection unit 32 may select the most recent three measurement values in response to the phrase “The major axis has increased by 5 mm compared to the previous time”.

The creation unit 34 creates a plot diagram P1 including the measurement values selected by the selection unit 32. That is, the plot diagram P1 is a line graph including the measurement values of the portion related to the comment on findings L1. The controller 36 controls the display 24 to display the screen D1 including the plot diagram P1 created by the creation unit 34.

According to the screen D1, the user can check the comment on findings L1 generated by the acquisition unit 30 and the plot diagram P1 including the measurement values of the portion related to the comment on findings L1. Therefore, it is possible to perform the work of creating an interpretation report while checking the plot diagram P1, which has better visibility than the plot diagram P0 (see FIG. 7) including all measurement values.

### Second Example

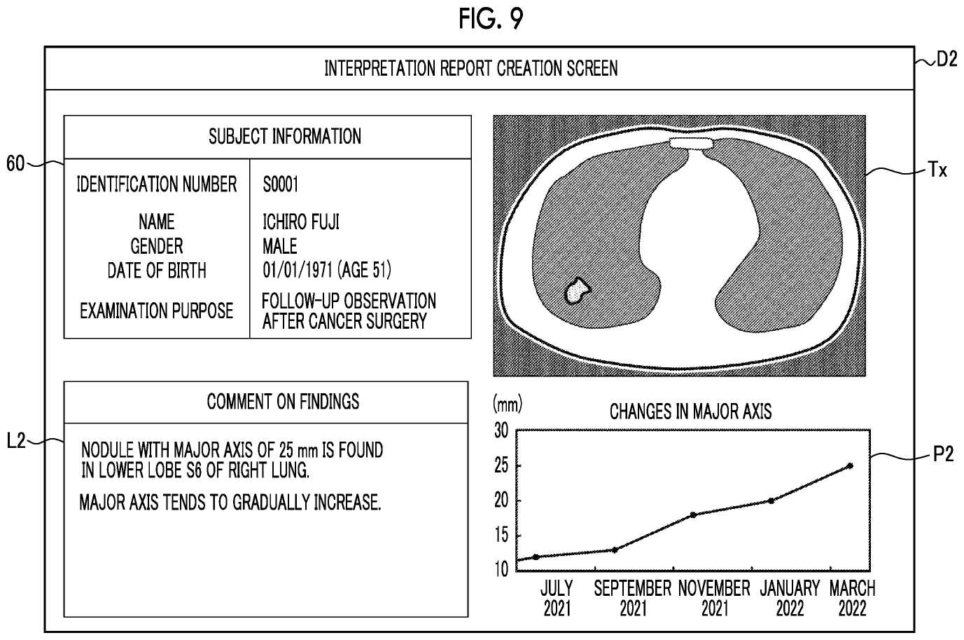

A second example will be described with reference to FIG. 9. FIG. 9 is an example of a screen D2 for creating an interpretation report, which is displayed on the display 24 by the controller 36. The screen D2 differs from the screen D1 of the first example in the contents of a comment on findings L2 and a plot diagram P2, but the rest is the same, so that redundant description will be omitted.

The comment on findings L2 includes a phrase that expresses changes over time in measurement values, such as “The major axis tends to gradually increase”. In a case where the user checks the measurement value corresponding to this phrase, it is assumed that the user will pay attention to the portion of the measurement value that tends to increase among all of the plurality of measurement values (see FIG. 7).

Therefore, as shown in the plot diagram P2, the selection unit 32 may select a measurement value of a portion with a large change in the direction of increase in response to the phrase “The major axis tends to gradually increase”. The portion with a large change may be, for example, a portion where the difference between two consecutive measurement values is equal to or greater than a predetermined threshold value. Further, the portion with a large change may be, for example, a portion where the difference between the maximum value and the minimum value in a predetermined range including two or more consecutive measurement values (for example, a range including five measurement values) is equal to or greater than a predetermined threshold value. In order to improve the visibility of the plot diagram P2, it is preferable to set the number of selected measurement values to about two to five times.

### Third Example

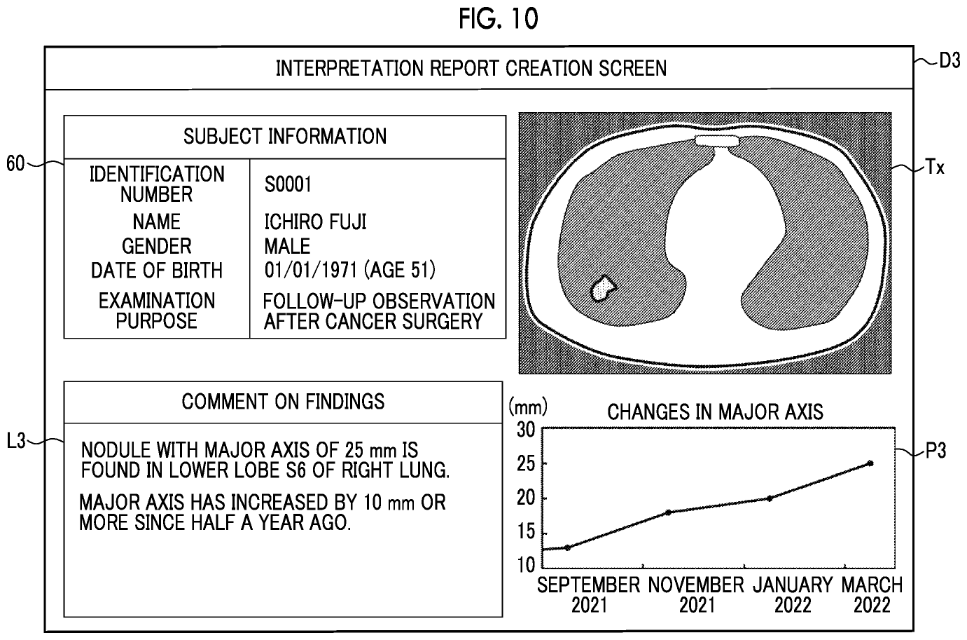

A third example will be described with reference to FIG. 10. FIG. 10 is an example of a screen D3 for creating an interpretation report, which is displayed on the display 24 by the controller 36. The screen D3 differs from the screen D1 of the first example in the contents of a comment on findings L3 and a plot diagram P3, but the rest is the same, so that redundant description will be omitted.

The comment on findings L3 includes a phrase that expresses changes over time in measurement values, such as “The major axis has increased by 10 mm or more since half a year ago”. In a case where the user checks the measurement value corresponding to this phrase, it is assumed that the user will pay attention to the measurement value half a year ago and the latest measurement value among all the plurality of measurement values (see FIG. 7).

Therefore, as shown in the plot diagram P3, the selection unit 32 may select measurement values from half a year ago to the most recent one in response to the phrase “The major axis has increased by 10 mm or more since half a year ago”. That is, the selection unit 32 may select a measurement value to which time information indicating a point in time of measurement determined based on a phrase related to the measurement value is added.

### Fourth Example

The selection unit 32 may select at least some of the plurality of measurement values acquired by the acquisition unit 30 based on a phrase that expresses the disease name included in the sentence acquired by the acquisition unit 30. That is, the selection unit 32 may vary the method of selecting the measurement value according to the phrase that expresses the disease name included in the sentence. This is because there are cases where the measurement value at which point in time should be paid attention to depends on the content of the disease.

For example, the selection unit 32 may select the most recent two measurement values in a case where the sentence acquired by the acquisition unit 30 includes the phrase that expresses the disease name, “diffuse panbronchiolitis”, and may select all of the plurality of measurement values in a case where the sentence includes the phrase “pneumonia”.

### Fifth Example

The selection unit 32 may select at least some of the plurality of measurement values acquired by the acquisition unit 30 based on a phrase that expresses the purpose of examination included in the sentence acquired by the acquisition unit 30. That is, the selection unit 32 may vary the method of selecting the measurement value according to the phrase that expresses the purpose of examination included in the sentence. This is because there are cases where the measurement value at which point in time should be paid attention to depends on the content of the examination.

For example, the selection unit 32 may select the most recent five measurement values in a case where the sentence acquired by the acquisition unit 30 includes the phrase that expresses the purpose of examination, “regular health checkup”, and may select the most recent three measurement values in a case where the sentence includes the phrase “postoperative follow-up observation”.

### Sixth Example

As shown in the comment on findings L1 in FIG. 8, the sentence acquired by the acquisition unit 30 may include a phrase (“major axis 25 mm”) that expresses the absolute value of the measurement value. In this case, the selection unit 32 may determine whether to select the measurement value based on the result of comparison between the measurement value included in the sentence and a predetermined threshold value. For example, in a case where the sentence includes a phrase that expresses a measurement value that is equal to or greater than a predetermined threshold value for measurement values meaning that the medical condition is bad in proportion to the magnitude of the numerical value, the selection unit 32 may select the measurement value.

On the other hand, in a case where the sentence includes a phrase that expresses a measurement value that is less than the predetermined threshold value, the selection unit 32 does not have to select the measurement value. Further, in a case where none of the measurement values is selected by the selection unit 32, the creation unit 34 may or may not create a plot diagram including all of the plurality of measurement values acquired by the acquisition unit 30. This is because in a case where none of the measurement values is selected by the selection unit 32, there is a likelihood that there is no measurement value of interest.

### Seventh Example

The sentences acquired by the acquisition unit 30 may include a plurality of phrases (“major axis 20 mm”, “major axis 25 mm”) that express the absolute value of the measurement value, such as “The major axis was 20 mm in the previous time, but the major axis increased to 25 mm in this time”. In this case, the selection unit 32 may determine whether to select at least two measurement values based on the result of comparison between the difference between the at least two measurement values included in the sentence and a predetermined threshold value.

For example, the selection unit 32 may select two measurement values included in the sentence in a case where the difference between the two measurement values is equal to or greater than a predetermined threshold value and indicates that the variation is large. Further, for example, in a case where the sentence includes three or more measurement values, the selection unit 32 may select the three or more measurement values in a case where the difference between the maximum value and the minimum value among the three or more measurement values is equal to or greater than a predetermined threshold value and indicates that the variation is large.

### Eighth Example

In the first to third examples (see FIGS. 8 to 10), an example of a form in which the selection unit 32 selects at least some of a plurality of measurement values that are continuous in time series order has been described, but the present disclosure is not limited thereto. In the present example, an example of a form in which the selection unit 32 selects at least some of a plurality of measurement values that are discrete in time series order will be described.

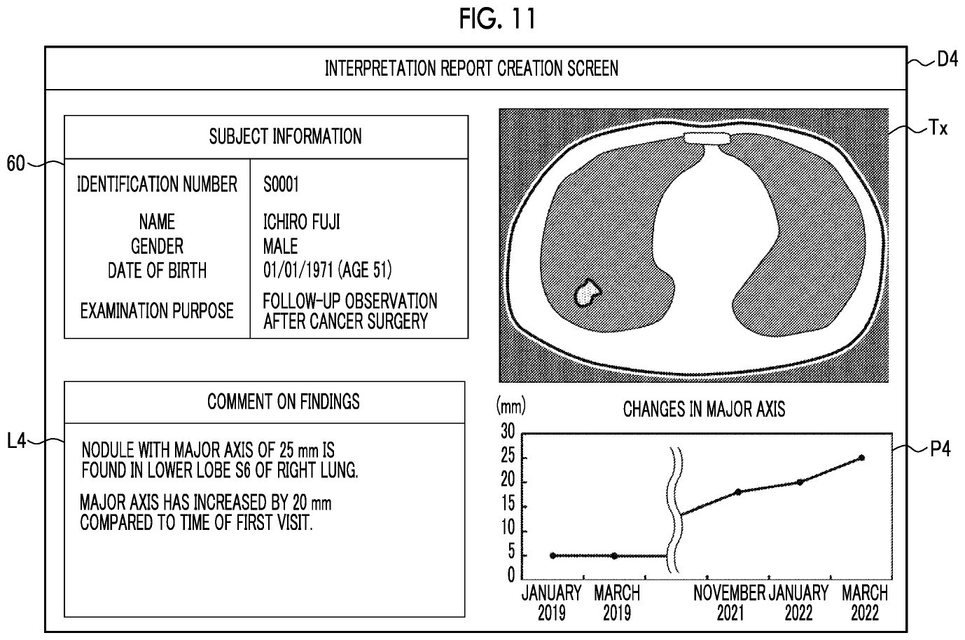

An eighth example will be described with reference to FIG. 11. FIG. 11 is an example of a screen D4 for creating an interpretation report, which is displayed on the display 24 by the controller 36. The screen D4 differs from the screen D1 of the first example in the contents of a comment on findings L4 and a plot diagram P4, but the rest is the same, so that redundant description will be omitted.

The comment on findings L4 includes a phrase that expresses changes over time in measurement values, such as “The major axis has increased by 20 mm compared to the time of the first visit”. In a case where the user checks the measurement value corresponding to this phrase, it is assumed that the user will pay attention to the measurement value at the time of the first visit and the latest measurement value among all the plurality of measurement values (see FIG. 7).

Therefore, as shown in the plot diagram P4, the selection unit 32 may select the first two measurement values (that is, at the time of the first visit), the most recent three measurement values, and five measurement values that are discrete in time series order in response to the phrase “The major axis has increased by 20 mm compared to the time of the first visit”. In this case, the creation unit 34 may create the plot diagram P4 using an omitting line (wavy line) indicating that the intermediate measurement values are omitted.

### Ninth Example

In the above example, a form in which the selection unit 32 selects some of a plurality of measurement values based on various phrases related to the measurement values included in the sentence has been described, but the present disclosure is not limited thereto. The selection unit 32 may additionally select a measurement value in addition to the measurement value selected based on the phrase related to the measurement value. For example, in a case where the difference between at least two measurement values included in the plurality of measurement values satisfies a predetermined condition, the selection unit 32 may select the at least two measurement values.

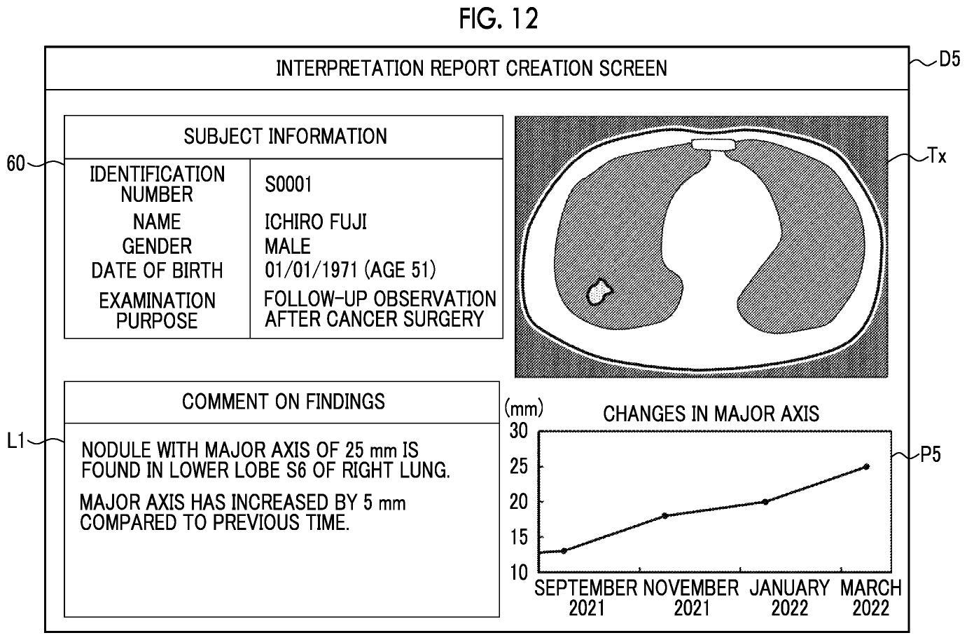

A ninth example will be described with reference to FIG. 12. The ninth example is a modification example of the first example, and is an example of a form in which a measurement value as of September 2021 is also selected in addition to the most recent three measurement values selected based on the phrase “The major axis has increased by 5 mm compared to the previous time” included in the comment on findings L1. FIG. 12 is an example of a screen D5 for creating an interpretation report, which is displayed on the display 24 by the controller 36. The screen D5 differs from the screen D1 of the first example in the contents of a plot diagram P5, but the other elements including the comment on findings L1 are the same, so that redundant description will be omitted.

As in the first example, the selection unit 32 first selects the most recent three measurement values in response to the phrase “The major axis has increased by 5 mm compared to the previous time” included in the comment on findings L1. After that, the selection unit 32 may select a portion with a large variation such that the difference between two consecutive measurement values from among the plurality of measurement values is equal to or greater than a predetermined threshold value. In a case where the threshold value is set to 5 in the example of FIG. 6, the difference between the measurement value as of September 2021 and the measurement value as of November 2021 immediately after that is 5. Therefore, the selection unit 32 may additionally select the measurement value as of September 2021 in addition to the most recent three measurement values.

Further, for example, the selection unit 32 may select a portion with a large variation such that the difference between the maximum value and the minimum value in a predetermined range including two or more consecutive measurement values (for example, a range including five measurement values) is equal to or greater than a predetermined threshold value.

According to such a form, even though there is no description in the sentence, measurement values of the portion with a large variation can be included in the plot diagram and presented. That is, since it is possible to present a plot diagram including measurement values at the point in time at which it is suspected that the medical condition has suddenly deteriorated or improved, it is possible to prevent oversight.

### Tenth Example

Similar to the ninth example, the selection unit 32 may further select a measurement value that satisfies a predetermined condition from among the plurality of measurement values, in addition to the measurement values selected based on various phrases related to the measurement value. For example, the selection unit 32 may select measurement values that are equal to or greater than a predetermined threshold value for measurement values meaning that the medical condition is bad in proportion to the magnitude of the numerical value.

According to such a form, even though there is no description in the sentence, measurement values having a particularly bad value can be included in the plot diagram and presented. That is, since it is possible to present a plot diagram including measurement values at the point in time at which it is suspected that the medical condition is particularly bad, it is possible to prevent oversight.

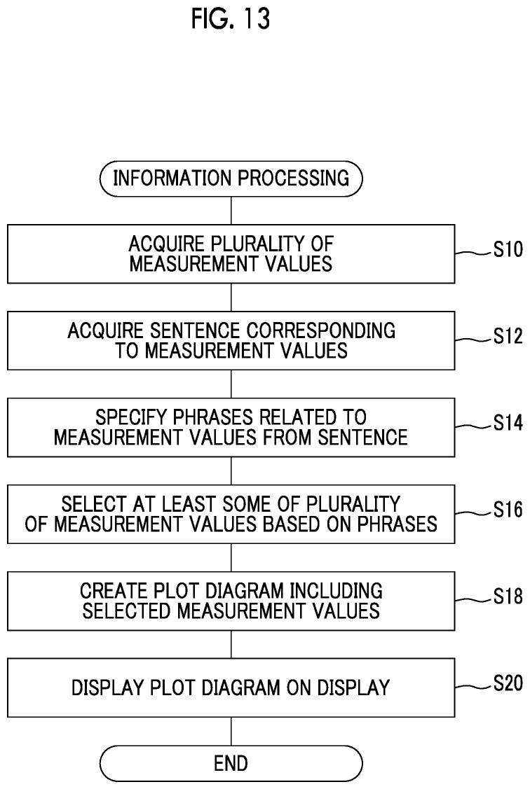

Next, with reference to FIG. 13, operations of the information processing apparatus 10 according to the present embodiment will be described. In the information processing apparatus 10, as the CPU 21 executes the information processing program 27, information processing shown in FIG. 13 is executed. The information processing is executed, for example, in a case where the user gives an instruction to start execution via the input unit 25.

In Step S10, the acquisition unit 30 acquires a plurality of measurement values measured from the same subject at a plurality of different points in time. In Step S12, the acquisition unit 30 acquires a sentence corresponding to the measurement values acquired in Step S10. In Step S14, the selection unit 32 specifies phrases corresponding to the measurement values from the sentence acquired in Step S12. In Step S16, the selection unit 32 selects at least some of the plurality of measurement values acquired in Step S10 based on the phrases corresponding to the measurement values specified in Step S14.

In Step S18, the creation unit 34 creates a plot diagram including at least some of the measurement values selected in Step S16. In Step S20, the controller 36 controls the display 24 to display the plot diagram created in Step S18, and ends this information processing.

As described above, the information processing apparatus 10 according to one aspect of the present disclosure comprises at least one processor, and the processor is configured to: acquire a plurality of measurement values measured from the same subject at a plurality of different points in time; acquire a sentence corresponding to the measurement value; and select at least some of the plurality of measurement values based on a phrase related to the measurement value included in the sentence.

That is, with the information processing apparatus 10 according to the present embodiment, it is possible to selectively present a measurement value assumed to attract the user's attention among a plurality of measurement values. Therefore, measurement values can be presented in a form with an excellent visibility in the work of creating an interpretation report or the like, and the creation of medical documents can be supported.

In addition, in the above-described embodiment, a form in which the acquisition unit 30 derives a measurement value by performing image analysis on a medical image has been described, but the present disclosure is not limited thereto. For example, the acquisition unit 30 may acquire measurement values stored in advance in the storage unit 22, the image server 5, the image DB 6, the report server 7, the report DB 8, and other external devices. Alternatively, for example, the acquisition unit 30 may acquire a measurement value manually input by the user via the input unit 25.

Further, in the above-described embodiment, a form in which the acquisition unit 30 generates a sentence corresponding to a measurement value from a medical image by machine learning has been described, but the present disclosure is not limited thereto. For example, the acquisition unit 30 may acquire sentences stored in advance in the report DB 8, the storage unit 22, and other external devices. Alternatively, for example, the acquisition unit 30 may acquire a sentence manually input by the user via the input unit 25.

Further, in the above-described embodiment, the measurement value representing the major axis of one lesion was used for description, but the present disclosure is not limited thereto. For example, in a case where there are a plurality of lesions in the same subject, the acquisition unit 30 may acquire measurement values at a plurality of points in time for each of the plurality of lesions, and the selection unit 32 may select some measurement values for each of the plurality of lesions. Further, for example, the acquisition unit 30 may acquire a plurality of types of measurement values (e.g., major axis and signal value) at a plurality of points in time for the same lesion, and the selection unit 32 may select some measurement values for each of a plurality of types of measurement values. In these cases, the creation unit 34 may create a single plot diagram by combining measurement values for a plurality of lesions and/or a plurality of types of measurement values.

Also, a user who has checked a plot diagram including some measurement values created in the above-described embodiment may then desire to check a plot diagram including all measurement values (see FIG. 7). Therefore, the controller 36 may receive a user's instruction to display a plot diagram including all measurement values via the input unit 25. Further, the creation unit 34 may create a plot diagram for a plurality of measurement values in a case where the controller 36 receives an instruction to display a plot diagram including all measurement values. The controller 36 may control the display 24 to display a plot diagram including all the measurement values created by the creation unit 34 instead of or in addition to a plot diagram including some of the measurement values.

Further, in the above-described embodiment, a form has been described assuming a situation in which an interpretation report is created in the interpretation WS 3, but the present disclosure is not limited thereto. For example, the information processing apparatus 10 may present a plot diagram selectively including some of the plurality of measurement values based on sentences included in the interpretation report to be viewed in a situation in which the interpretation report is viewed in the interpretation WS 3 and/or the medical care WS 4. According to such a form, the plot diagram can be presented in a form with an excellent visibility to the viewer of the interpretation report, and the visibility of the interpretation report can be improved regardless of what kind of plot diagram the creator was checking in the situation of creating the interpretation report.

Further, in the above-described embodiment, a form assuming an interpretation report for medical images has been described, but the present disclosure is not limited thereto. The information processing apparatus 10 according to the present disclosure is applicable to creating and/or viewing various medical documents including sentences and measurement values. For example, the information processing apparatus 10 may be applied to creating and/or viewing a report on the results of regular health checkups.

In the above embodiments, for example, as hardware structures of processing units that execute various kinds of processing, such as the acquisition unit 30, the selection unit 32, the creation unit 34, and the controller 36, various processors shown below can be used. As described above, the various processors include a programmable logic device (PLD) as a processor of which the circuit configuration can be changed after manufacture, such as a field programmable gate array (FPGA), a dedicated electrical circuit as a processor having a dedicated circuit configuration for executing specific processing such as an application specific integrated circuit (ASIC), and the like, in addition to the CPU as a general-purpose processor that functions as various processing units by executing software (program).

One processing unit may be configured by one of the various processors, or may be configured by a combination of the same or different kinds of two or more processors (for example, a combination of a plurality of FPGAs or a combination of the CPU and the FPGA). In addition, a plurality of processing units may be configured by one processor.

As an example in which a plurality of processing units are configured by one processor, first, there is a form in which one processor is configured by a combination of one or more CPUs and software as typified by a computer, such as a client or a server, and this processor functions as a plurality of processing units. Second, as represented by a system on chip (SoC) or the like, there is a form of using a processor for realizing the function of the entire system including a plurality of processing units with one integrated circuit (IC) chip. In this way, various processing units are configured by one or more of the above-described various processors as hardware structures.

Furthermore, as the hardware structure of the various processors, more specifically, an electrical circuit (circuitry) in which circuit elements such as semiconductor elements are combined can be used.

In the above embodiment, the information processing program 27 is described as being stored (installed) in the storage unit 22 in advance; however, the present disclosure is not limited thereto. The information processing program 27 may be provided in a form recorded in a recording medium such as a compact disc read only memory (CD-ROM), a digital versatile disc read only memory (DVD-ROM), and a universal serial bus (USB) memory. In addition, the information processing program 27 may be downloaded from an external device via a network. Further, the technique of the present disclosure extends to a storage medium for storing the information processing program non-transitorily in addition to the information processing program.

The technique of the present disclosure can be appropriately combined with the above-described embodiments and examples. The described contents and illustrated contents shown above are detailed descriptions of the parts related to the technique of the present disclosure, and are merely an example of the technique of the present disclosure. For example, the above description of the configuration, function, operation, and effect is an example of the configuration, function, operation, and effect of the parts according to the technique of the present disclosure. Therefore, needless to say, unnecessary parts may be deleted, new elements may be added, or replacements may be made to the described contents and illustrated contents shown above within a range that does not deviate from the gist of the technique of the present disclosure.

What is claimed is:

1. An information processing apparatus comprising at least one processor, wherein the at least one processor is configured to:
acquire a plurality of measurement values measured from the same subject at a plurality of different points in time;
acquire a sentence corresponding to the measurement value; and
select at least some of the plurality of measurement values based on a phrase related to the measurement value included in the sentence.

2. The information processing apparatus according to claim 1, wherein the at least one processor is configured to select at least some of the plurality of measurement values based on a phrase that expresses a change over time in the measurement value included in the sentence.

3. The information processing apparatus according to claim 1, wherein:
time information indicating a point in time of measurement is added to the measurement value, and
the at least one processor is configured to:
create a plot diagram including the at least some selected measurement values using the measurement value and the time information as variables; and
cause a display to display the plot diagram.

4. The information processing apparatus according to claim 3, wherein the at least one processor is configured to, in a case where an instruction is received:
create the plot diagram including all of the plurality of acquired measurement values; and
cause the display to display the plot diagram.

5. The information processing apparatus according to claim 1, wherein:
time information indicating a point in time of measurement is added to the measurement value, and
the at least one processor is configured to select the measurement value to which the time information indicating the point in time of measurement determined based on the phrase related to the measurement value is added.

6. The information processing apparatus according to claim 1, wherein the at least one processor is configured to select at least some of the plurality of measurement values according to the number of measurement values determined based on the phrase related to the measurement value.

7. The information processing apparatus according to claim 1 wherein the at least one processor is configured to select at least some of the plurality of measurement values based on a phrase that expresses a disease name included in the sentence.

8. The information processing apparatus according to claim 1, wherein the at least one processor is configured to select at least some of the plurality of measurement values based on a phrase that expresses a purpose of examination included in the sentence.

9. The information processing apparatus according to claim 1, wherein the at least one processor is configured to determine whether to select the measurement value based on a result of comparison between the measurement value included in the sentence and a predetermined threshold value.

10. The information processing apparatus according to claim 1, wherein the at least one processor is configured to determine whether to select at least two measurement values included in the sentence based on a result of comparison between a difference between the at least two measurement values and a predetermined threshold value.

11. The information processing apparatus according to claim 1, wherein the at least one processor is configured to select the measurement value that satisfies a predetermined condition from among the plurality of measurement values.

12. The information processing apparatus according to claim 1, wherein the at least one processor is configured to, in a case where a difference between at least two measurement values included in the plurality of measurement values satisfies a predetermined condition, select the at least two measurement values.

13. The information processing apparatus according to claim 1, wherein:
time information indicating a point in time of measurement is added to the measurement value, and
the at least one processor is configured to select at least some of the plurality of measurement values that are continuous in time series order.

14. The information processing apparatus according to claim 1, wherein:
time information indicating a point in time of measurement is added to the measurement value, and
the at least one processor is configured to select at least some of the plurality of measurement values that are discrete in time series order.

15. The information processing apparatus according to claim 1, wherein the measurement value is at least one of a size of a lesion or a signal value at a part of the lesion in a medical image obtained by imaging the lesion.

16. An information processing method comprising:
acquiring a plurality of measurement values measured from the same subject at a plurality of different points in time;
acquiring a sentence corresponding to the measurement value; and
selecting at least some of the plurality of measurement values based on a phrase related to the measurement value included in the sentence.

17. A non-transitory computer-readable storage medium storing an information processing program for causing a computer to execute a process comprising:
acquiring a plurality of measurement values measured from the same subject at a plurality of different points in time;
acquiring a sentence corresponding to the measurement value; and
selecting at least some of the plurality of measurement values based on a phrase related to the measurement value included in the sentence.
